(19) United States
(12) Patent Application Publication
(10) Pub. No.: US 2023/0364175 A1
(43) Pub. Date: Nov. 16, 2023

(54) HERBAL COMPOSITIONS AND METHODS OF TREATMENT USING THE SAME

(71) Applicants: April Brader, St. Petersburg Beach, FL (US); Joel Snook, St. Petersburg Beach, FL (US); Yohann Samarasinghe, La Jolla, CA (US); Samarth Aggarwal, San Diego, CA (US)

(72) Inventors: April Brader, St. Petersburg Beach, FL (US); Joel Snook, St. Petersburg Beach, FL (US); Yohann Samarasinghe, La Jolla, CA (US); Samarth Aggarwal, San Diego, CA (US)

(21) Appl. No.: 18/316,182

(22) Filed: May 11, 2023

Related U.S. Application Data
(60) Provisional application No. 63/364,519, filed on May 11, 2022.

(51) Int. Cl. A61K 36/886; A61K 36/355; A61K 36/19; A61K 31/4045; A61K 31/724; A61K 9/00; A61P 11/02; A61P 29/00

(52) U.S. Cl. CPC A61K 36/886; A61K 36/355; A61K 36/19; A61K 31/4045; A61K 31/724; A61K 9/0043; A61P 11/02; A61P 29/00

(57) ABSTRACT

An herbal composition including Lonicera Japonica and Andrographis Paniculata, a method of preparing the herbal composition, and a method of administering the herbal composition to a subject in need thereof is disclosed. The method of administering can include intranasal administration.

### CROSS-REFERENCE TO RELATED APPLICATIONS

This application claims the benefit of priority under 35 U.S.C. § 119(e) of U.S. Provisional Application Ser. No. 63/364,519 filed on May 11, 2022, the content of which is relied upon and incorporated herein by reference in its entirety.

### FIELD

The present disclosure generally relates to herbal compositions, methods of preparing the herbal compositions, and methods of treating a subject with the herbal compositions; and more particularly, to herbal compositions formulated for intranasal delivery (e.g., nano spray), methods of preparing the formulations for intranasal delivery, and methods of administering the formulations to treat respiratory or inflammatory conditions, and/or viral, bacterial, or fungal infections, in a subject in need thereof.

### BACKGROUND

Existing treatments for conditions, infections, and ailments caused by infectious diseases, such as the coronavirus, have proven to be effective for some subjects but not others. Accordingly, there is a need for additional treatments for respiratory conditions, inflammatory conditions, viral infections, bacterial infections, and fungal infections.

### SUMMARY

In various embodiments, an herbal composition is provided. The herbal composition can comprise Lonicera Japonica and Andrographis Paniculata; wherein each of the Lonicera Japonica and Andrographis Paniculata is included, individually, in an amount ranging from about 0.01% to about 5% w/w.

In some embodiments, each of the Lonicera Japonica and Andrographis Paniculata is included, individually, in an amount ranging from about 0.1% to about 1% w/w.

In some embodiments, the Lonicera Japonica and Andrographis Paniculata have a combined concentration of 0.666% (w/w).

In some embodiments, the Lonicera Japonica and Andrographis Paniculata are included in a concentration ratio in the range of 0.2 to 5.0.

In some embodiments, the herbal composition further comprises melatonin.

In some embodiments, the herbal composition further comprises aloe vera.

In some embodiments, the herbal composition further comprises hydroxypropyl-beta-cyclodextrin.

In some embodiments, the herbal composition further comprises melatonin; wherein the melatonin has a concentration of 0.052% (w/w) and the hydroxypropyl-beta-cyclodextrin has a concentration of 0.520% (w/w).

In some embodiments, the herbal composition is formulated for intranasal administration.

In various embodiments, a method of treating a respiratory or inflammatory condition in a subject in need thereof is provided. The method can comprise administering an herbal composition to the subject, the herbal composition comprising Lonicera Japonica and Andrographis Paniculata; wherein each of the Lonicera Japonica and Andrographis Paniculata is included, individually, in an amount ranging from about 0.01% to about 5% w/w.

In some embodiments of the method, each of the Lonicera Japonica and Andrographis Paniculata is included, individually, in an amount ranging from about 0.1% to about 1% w/w.

In some embodiments of the method, the Lonicera Japonica and Andrographis Paniculata have a combined concentration of 0.666% (w/w).

In some embodiments of the method, the Lonicera Japonica and Andrographis Paniculata are included in a concentration ratio in the range of 0.2 to 5.0.

In some embodiments of the method, the herbal composition further comprises aloe vera.

In some embodiments of the method, the herbal composition further comprises melatonin.

In some embodiments of the method, the herbal composition further comprises hydroxypropyl-beta-cyclodextrin.

In some embodiments of the method, the herbal composition further comprises melatonin; wherein the melatonin has a concentration of 0.052% (w/w) and the hydroxypropyl-beta-cyclodextrin has a concentration of 0.520% (w/w).

In some embodiments of the method, the herbal composition further comprises hydroxypropyl-beta-cyclodextrin.

In some embodiments of the method, the melatonin has a concentration of 0.052% (w/w) and the hydroxypropyl-beta-cyclodextrin has a concentration of 0.520% (w/w).

In various embodiments, a method of treating a respiratory or inflammatory condition in a subject in need thereof is provided. The method can comprise administering an herbal composition to the subject, the herbal composition comprising Lonicera Japonica and Andrographis Paniculata; wherein each of the Lonicera Japonica and Andrographis Paniculata is included, individually, in an amount ranging from about 0.01% to about 5% w/w; wherein the Lonicera Japonica and Andrographis Paniculata are included in a concentration ratio in the range of 0.2 to 5.0; and wherein the administering is intranasal administration.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein and, together with the description, explain the principles and operations of the claimed subject matter.

### DETAILED DESCRIPTION

Reference will now be made in detail to the exemplary embodiment(s), examples of which is/are illustrated in the examples. Before describing the exemplary embodiments, it is noted the embodiments reside primarily in combinations of components and procedures related to the herbal composition. Accordingly, the composition and method components have been represented where appropriate, showing only those specific details that are pertinent to understanding the embodiments of the present disclosure so as not to obscure the disclosure with details that will be readily apparent to those of ordinary skill in the art having the benefit of the description herein. The specific details of the various embodiments described herein are used for demonstration purposes only, and no unnecessary limitation or inferences are to be understood therefrom.

In various embodiments, an herbal composition, a process for the preparation of the herbal composition, and a process for the administration of the herbal composition to a subject are provided. In some embodiments, the herbal composition disclosed herein possesses anti-viral, anti-fungal, anti-toxin, anti-bacterial, anti-parasitic, and/or anti-biofilm properties. The herbal composition, in various embodiments, may be used to treat one or more respiratory or inflammatory conditions in a subject. In some embodiments, the herbal composition may be used to treat one or more viral, bacterial, or fungal infections.

In some embodiments, the herbal composition comprises one or more herb components. As used herein, an herb component may include matter derived from a plant or plant part and used for consumption, including, for example, for medicinal, therapeutic, aromatic, and/or culinary purposes. In some embodiments, the matter derived from a plant can be obtained from one or more parts of the plant, including, for example, the whole fruit, whole vegetable, whole plant, whole tree, whole bush, seed, peel, fruit, stem, bark, leaf, root, flower, petal, bulb, etc.

Extracts from one or more species of the one or more herb components can be obtained by extraction methods known to those of ordinary skill in the art. In some embodiments, the extract can be obtained from one or more portions of the plant, including, for example, the whole fruit, whole vegetable, whole plant, whole tree, whole bush, seed, peel, fruit, stem, bark, leaf, root, flower, petal, bulb, etc.

In some embodiments, the herbal composition comprises the parts, extracts, or isolated compounds (alone or collectively, “herb components”) from one or more plants, two or more plants, or a plurality of plants. In some embodiments, the herbal composition comprises one or more herb components obtained from plant components or extracts therefrom, including, for example, Lonicera Japonica (Jin Yin Hua), Andographis Paniculata (Chuan Xin Lian), Hedyotis Diffusae Herba (Bai Hua She She Cao), Patriniae Herba (Bai Jiang Cao), Pulsatilla Radix (Bai Tou Weng), Dictamnus Root Bark (Bai Xian Pi), Isatidis/Baphicacanthis (Ban Lan Gen), Scutellariae Barbatae Herba (Ban Zhi Lian), Paridis Rhizoma (Chong Lou), Isatidis Follium (Da Qing Ye), Hypericum Perforatum (Guan Ye Lian Qiao St John's Wort), Nelumbinis Folium (He Ye), Sargentodoxae Caulis (Hong Teng), Begoniae Fimristipulatae (Hong Tian Kui), Cortex Phellodendri (Huang Bai), Rhizoma Coptidis (Huang Lian), Radix Scutellariae (Huang Qin), Centellae Herba (Ji Xue Cao), Herba Lygodii Japonici (Jin Sha Teng), Nelumbinis Caulis (Lian Geng), Forsythiae Fructus (Lian Qiao), Rhapontici Radix (Lou Lu), Phaseoli Radiati Semen (Lu Dou), Lasiosphaera Calvatia (Ma Bo), Portulacae Herba (Ma Chi Xian), Taraxaci Herba (Pu Gong Ying Dandelion), Indigo Naturalis (Qing Dai), Lonicerae Caulis (Ren Dong Teng Honeysuckle Vine), Cremastrae/Pleiones Pseudobulbus (Shan Ci Gu), Sophorae Tonkinensis Radix (Shan Dou Gen), Belamcandae Rhizome (She Gan), Follium Nelumbis Nuciferae (Sheng He Ye), Luffae Fructus Retinervus (Si Gua Luo), Semiaquilegiae Radix (Tian Kui Zi), Smilacis Glabrae Rhizoma (Tu Fu ling), Achyranthis (Tu Niu Xi), Radix Scrophularia (Xuan Shen), Brucae Fructus (Ya Dan Zi), Chrysanthemi Indici Flos (Ye Jua Hua), Houttuyniae Herba (Yu Xing Cao), Rhizoma Paridis (Zao Xiu), Violae Herba (Zi Hua Di Ding), Gardenias Fructus (Zhi Zi), Hippophae ramnoides (Seabuckthorne fruit oil), Gigartina skottsbergi (red algae), Padina tetrastromatica (brown algae), Gracilaria lemaneiformis (Red algae), Arthrospira platensis, Erodium glaucophyllum (Storkbill), Sargassum henslowianum, Azadiractha indica (Neem), Dimocarpus longan (Longan fruit), Cistus incanus, Sutherlandia frutescens (Cancer Bush), Ocimum basilicum (Holy Basil), Salvia officinalis (Sage), Phyllanthus niruri (Chanca Piedra), Glycyrrhizae Radix (Licorice Root), Armeniacae Semum Amarum (Xing Ren), Miltiorrhizae Radix (Dan Shen), Platycodon grandifloras (Jie Geng), Ribes nigrum (Blackcurrant), Adansonia digitate (Baobab), Burke Africana, and Pelargonium sidoides (Umckaloabo). The listed herb components are known to have minimal or no side effects on human subjects.

In some embodiments, the herbal composition comprises Lonicera Japonica and one or more additional herb components selected from Andrographis Paniculata, Hedyotis Diffusae Herba, Patriniae Herba, Pulsatilla Radix, Dictamnus Root Bark, Isatidis/Baphicacanthis, Scutellariae Barbatae Herba, Paridis Rhizoma, Isatidis Follium, Hypericum Perforatum, Nelumbinis Folium, Sargentodoxae Caulis, Begoniae Fimristipulatae, Cortex Phellodendri, Rhizoma Coptidis, Radix Scutellariae, Centellae Herba, Herba Lygodii Japonici, Nelumbinis Caulis, Forsythias Fructus, Rhapontici Radix, Phaseoli Radiati Semen, Lasiosphaera Calvatia, Portulacae Herba, Taraxaci Herba, Indigo Naturalis, Lonicerae Caulis, Cremastrae/Pleiones Pseudobulbus, Sophorae Tonkinensis Radix, Belamcandae Rhizome, Follium Nelumbis Nuciferae, Luffae Fructus Retinervus, Semiaquilegiae Radix, Smilacis Glabrae Rhizoma, Achyranthis, Radix Scrophularia, Brucae Fructus, Chrysanthemi Indici Flos, Houttuyniae Herba, Rhizoma Paridis, Violae Herba, Gardenias Fructus, Hippophae ramnoides, Gigartina skottsbergi, Padina tetrastromatica, Gracilaria lemaneiformis, Arthrospira platensis, Erodium glaucophyllum, Sargassum henslowianum, Azadiractha indica, Dimocarpus longan, Cistus incanus, Sutherlandia frutescens, Ocimum basilicum, Salvia officinalis, Phyllanthus niruri, Glycyrrhizae Radix, Armeniacae Semum Amarum, Miltiorrhizae Radix, Platycodon grandifloras, Ribes nigrum, Adansonia digitate, Burke Africana, and Pelargonium sidoides, and Andrographis Paniculata.

In some embodiments, the herbal composition comprises Andrographis Paniculata and one or more additional herb components selected from Lonicera Japonica, Hedyotis Diffusae Herba, Patriniae Herba, Pulsatilla Radix, Dictamnus Root Bark, Isatidis/Baphicacanthis, Scutellariae Barbatae Herba, Paridis Rhizoma, Isatidis Follium, Hypericum Perforatum, Nelumbinis Folium, Sargentodoxae Caulis, Begoniae Fimristipulatae, Cortex Phellodendri, Rhizoma Coptidis, Radix Scutellariae, Centellae Herba, Herba Lygodii Japonici, Nelumbinis Caulis, Forsythias Fructus, Rhapontici Radix, Phaseoli Radiati Semen, Lasiosphaera Calvatia, Portulacae Herba, Taraxaci Herba, Indigo Naturalis, Lonicerae Caulis, Cremastrae/Pleiones Pseudobulbus, Sophorae Tonkinensis Radix, Belamcandae Rhizome, Follium Nelumbis Nuciferae, Luffae Fructus Retinervus, Semiaquilegiae Radix, Smilacis Glabrae Rhizoma, Achyranthis, Radix Scrophularia, Brucae Fructus, Chrysanthemi Indici Flos, Houttuyniae Herba, Rhizoma Paridis, Violae Herba, Gardeniae Fructus, Hippophae ramnoides, Gigartina skottsbergi, Padina tetrastromatica, Gracilaria lemaneiformis, Arthrospira platensis, Erodium glaucophyllum, Sargassum henslowianum, Azadiractha indica, Dimocarpus longan, Cistus incanus, Sutherlandia frutescens, Ocimum basilicum, Salvia officinalis, Phyllanthus niruri, Glycyrrhizae Radix, Armeniacae Semum Amarum, Miltiorrhizae Radix, Platycodon grandifloras, Ribes nigrum, Adansonia digitate, Burke Africana, and Pelargonium sidoides.

In some embodiments, the herbal composition comprises Andrographis Paniculata, Lonicera Japonica, and one or more additional herb components selected from Hedyotis Diffusae Herba, Patriniae Herba, Pulsatilla Radix, Dictamnus Root Bark, Isatidis/Baphicacanthis, Scutellariae Barbatae Herba, Paridis Rhizoma, Isatidis Follium, Hypericum Perforatum, Nelumbinis Folium, Sargentodoxae Caulis, Begoniae Fimristipulatae, Cortex Phellodendri, Rhizoma Coptidis, Radix Scutellariae, Centellae Herba, Herba Lygodii Japonici, Nelumbinis Caulis, Forsythias Fructus, Rhapontici Radix, Phaseoli Radiati Semen, Lasiosphaera Calvatia, Portulacae Herba, Taraxaci Herba, Indigo Naturalis, Lonicerae Caulis, Cremastrae/Pleiones Pseudobulbus, Sophorae Tonkinensis Radix, Belamcandae Rhizome, Follium Nelumbis Nuciferae, Luffae Fructus Retinervus, Semiaquilegiae Radix, Smilacis Glabrae Rhizoma, Achyranthis, Radix Scrophularia, Brucae Fructus, Chrysanthemi Indici Flos, Houttuyniae Herba, Rhizoma Paridis, Violae Herba, Gardeniae Fructus, Hippophae ramnoides, Gigartina skottsbergi, Padina tetrastromatica, Gracilaria lemaneiformis, Arthrospira platensis, Erodium glaucophyllum, Sargassum henslowianum, Azadiractha indica, Dimocarpus longan, Cistus incanus, Sutherlandia frutescens, Ocimum basilicum, Salvia officinalis, Phyllanthus niruri, Glycyrrhizae Radix, Armeniacae Semum Amarum, Miltiorrhizae Radix, Platycodon grandifloras, Ribes nigrum, Adansonia digitate, Burke Africana, and Pelargonium sidoides.

The one or more herb components of the herbal composition may be included in any suitable form. In some embodiments, for example, the one or more herb components are processed to form extracts, dried, powdered, pelleted, concentrated, gelled, etc. In some embodiments, the one or more herb components are dried and powdered, and then incorporated into the herbal composition. In some embodiments, for example, the one or more herb components are maintained as an aqueous extract, alcohol extract, or a combination of aqueous/alcohol extract. In some embodiments, one or more herb components of the herbal composition can be extracted with a suitable solvent (e.g., ethanol), combined with one or more additional herb components, and concentrated to a concentration suitable for the herbal composition. In some embodiments, the resulting concentrate of the herbal composition is dried to provide a powdery composition, which may be useful for dosing or storage of the herb components. In some embodiments, the one or more herb components are formulated for nebulization, vaporization, or another method of inhalation.

Parts, extracts, or isolated compounds of the one or more herb components may be combined in any suitable concentration ratio when forming the herbal composition. In some embodiments, for example, the herbal composition comprises two herb components. In such embodiments, each of the respective herb components can be extracted with a solvent and the extraction solutions can be combined to form an herbal composition in which the two herb components and solvent are present at a ratio of 0.5:0.5:10, or 1.0:1.0:10, or 1.5:1.5:10 (first herb component:second herb component:solvent). For example, in some embodiments, the herbal composition comprises Andrographis Paniculata and Lonicera Japonica, and the solvent is ethanol. In such an example, extracts of the herb components and ethanol can be combined in a ratio of 1.0:1.0:10 (Andrographis Paniculata to Lonicera Japonica to ethanol).

In some embodiments, the one or more herb components are combined in equal parts based on weight to obtain an organic mixture. The organic mixture can be included in the herbal composition in any suitable weight amount. In some embodiments, the herbal composition is provided to a user in a container useful for dispensing the composition, such as, for example, a nasal sprayer, misting device, aerosol device, etc. In some embodiments, the herbal composition includes the organic mixture in an amount ranging from about 0.25% to about 25%, or about 0.5% to about 15%, or about 1% to about 10%, or about 1% to about 8%, or about 2% to about 6%, or about 3% to about 5% (by weight). In some embodiments, each of the respective herb components are provided in the organic mixture in a range of about 1 mg to about 1000 mg, or about 10 mg to about 750 mg, or about 100 mg to about 500 mg, or about 300 mg to about 500 mg, or about 10 mg to about 250 mg, or about 15 mg to about 100 mg, etc., the amount for each being based on weight. For example, if the herbal composition includes the organic mixture in an amount of about 5%, such as 5 grams in a 100-gram herbal composition, and the organic mixture is composed of 10 specific herb components, then each of the ten herb components is provided in an amount of about 0.5 grams (500 mg).

In various embodiments, the one or more herbal components described herein may be combined in any suitable concentration ratio when forming the herbal composition. In some embodiments, for example, the composition comprises a plurality of herb components. In some embodiments, the composition comprises the plurality of herb components and one or more additional components. In some embodiments, each herb component is present in an equal amount (e.g., by weight), or a ratio of 1.0:1.0 respectively. In some embodiments, the respective herb components are present in different amounts. In some embodiments, two herb components are present at a ratio in the range of 0.10 to 10.0; 0.2 to 5.0; 0.5 to 2.5; or any range derivable therein (first herb component:second herb component). In some embodiments, two herb components are present at a ratio of 0.25:1.0; 0.5:1.0; 0.75:1.0; 1.0:1.0; 1.25:1.0; 1.50:1.0; 1.75:1.0; 2.0:1.0; etc. (first herb component:second herb component). In some embodiments, three or more herb components are present, and each respective component is present in an amount relative to one or each of the other components. For example, if the composition includes three herb components, the amount of the third component present may be expressed relative to the first component (1.0:1.0; third component:first component), the second component (1.0:0.5; third component:second component), or the first and second components (1.0:1.0:0.5; third component:first component:second component). In various embodiments, four or more, five or more, six or more, seven or more, eight or more, nine or more, ten or more, etc., herb components are present, and each respective herb component is present in an amount relative to one or each of the other components as described above.

In various embodiments, the composition comprises any number of combinations of the herb components. The concentrations of any herb components in the compositions can vary. In some embodiments, each of the respective herb components are provided in a range of from about 0.0001% to about 10%, or from about 0.001% to about 8%, or from about 0.01% to about 5%, or from about 0.1% to about 1%. In some embodiments, for example, the total amount for each respective herb component in the composition is about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.825%, 0.8500%, 0.8750%, 0.9000%, 0.9250%, 0.9500%, 0.9750%, 1.0%, 1.5%, 2.0%, 2.5%, 3.0%, 3.5%, 4.0%, 4.5%, 5%, 5.5%, 6.0%, 6.5%, 7.0%, 7.5%, 8.0%, 8.5%, 9.0%, 9.5%, or 10%; or any range derivable therein. The percentage can be calculated by weight or volume of the total composition. In some embodiments, the percentage is calculated by weight of the total composition. In some embodiments, the percentage is calculated by weight of the herbal mixture portion of the composition. In some embodiments, the concentrations vary depending on the addition, substitution, and/or subtraction of one or more herb components in a respective composition.

In some embodiments, the one or more herb components may include one or more flavonoids, terpenes, terpenoids, amino acids, proteins, sugars, enzymes, fatty acids, ester, and/or other organic compounds that are at least partially responsible for the beneficial activity of the herbal composition. In some embodiments, the herbal composition may include one or more additional (i.e., from a secondary source) flavonoids, terpenes, terpenoids, amino acids, proteins, sugars, enzymes, fatty acids, ester, and/or other organic compounds.

In some embodiments, the herbal composition further comprises one or more additional plant components or extracts. In some embodiments, for example, the one or more additional plant components or extracts includes a species of aloe, such as Aloe vera (also known as A. barbadensis Mill., A. indica Royle, A. perfoliate L. var., A. vugaris Lam.). In some embodiments, the one or more additional plant components or extracts includes a species of cactus, slippery elm, marshmallow root, seaweed, coconut oil, flaxseed oil, agave, soy, olive oil, walnut oil, calendula, cucumber juice, rose hip gel, etc.

In some embodiments, the herbal composition comprises one or more inorganic materials, including, elemental metals and minerals. In some embodiments, for example, the herbal composition comprises magnesium, magnesium salts, or magnesium complexes.

In some embodiments, the herbal composition comprises one or more organic compounds. In some embodiments, the one or more organic compounds includes melatonin. In such embodiments, the melatonin is provided in a suitable effective amount. In some embodiments, the melatonin is provided in a concentration ranging from about 0.049 to about 0.055 wt. %, including, for example, 0.050%, 0.051%, 0.052%, 0.053%, and 0.054%.

In some embodiments, the one or more organic compounds includes cyclodextrin or a cyclodextrin derivative. In some embodiments, the one or more organic compounds includes hydroxypropyl-beta-cyclodextrin (HPBCD). In some embodiments, HPBCD is provided in a concentration ranging from about 0.49 to about 0.55 wt. %, including, for example, 0.50%, 0.51%, 0.52%, 0.53%, and 0.54%. HPBCD has been found to be beneficial to the herbal composition for one or more reasons. In some embodiments, for example, HPBCD in the concentration range from about 0.49 to about 0.55 wt. % was found to improve the solubility of melatonin, which is relatively low (about 0.1 mg/mL, log P=1.6). The low solubility of melatonin can lead to limited absorption and bioavailability. With the addition of HPBCD, the solubility of melatonin is improved to about 10 mg/mL. In an aqueous solution in which HPBCD is present at a concentration ranging from about 0.49 to about 0.55 wt. %, HPBCD monomers have a hydrodynamic size of 10 nm and aggregates of HPBCD form up to 100 nm in diameter (measured via dynamic light scattering).

The herbal composition may be adapted for administration by any appropriate route, including, for example, nasal, aerosol, nebulizer solution, oral, rectal, topical, vaginal, and parenteral methods. In some embodiments, the herbal composition may be delivered through a surface of the subject's body; i.e., transdermally or transmucosally. As used herein, “transdermal” means passage into and through the skin to achieve effective therapeutic blood levels. As used herein, “transmucosal” means passage through a mucosal membrane of a living organism, and therefore includes delivery of through nasal, lungs, sublingual, oral patch, or buccal tissue. Transdermal or transmucosal delivery will involve topical application of the herbal composition in the form of an ointment, gel, cream, powder, spray, aerosol, nebulizer solution, or the like, or may involve use of a drug delivery device. The herbal compositions may optionally contain a permeation enhancer to increase the rate at which the herbal composition permeates through the skin or mucosal tissue.

In some embodiments, transmucosal administration of the herbal composition is achieved transnasally. In some embodiments, transmucosal administration is achieved by way of a nasal spray. Exemplary vehicles for use in a nasal spray include sterile saline solutions having a pH compatible with that of the nasal mucosa.

Solid nasal powders or insufflations can also be used for transmucosal delivery. For such purpose, the herbal composition is administered in finely divided solid form together with a pharmaceutically acceptable carrier. Exemplary carriers include a finely divided polyethylene glycol, finely divided lactose, and/or other finely divided excipients.

In some embodiments, the herbal composition may be formulated in the form of oral preparations such as powders, granules, tablets, capsules, suspensions, emulsions, syrups, aerosols, or the like, external preparations, suppositories, and sterilized injection solutions according to an existing method. Specific examples of carriers, excipients, and diluents that can be included in the composition may include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, talc, magnesium stearate, methylhydroxybenzoate, propylhydroxybenzoate, mineral oil, and the like.

In some embodiments, a solid formulation for oral administration may include tablets, pills, powders, granules, capsules, and the like. In such embodiments, the solid formulation may include at least one excipient such as starch, calcium carbonate, sucrose or lactose, and gelatin, in addition to the herbal composition. In addition to simple excipients, lubricants such as magnesium stearate and talc may also be included. A liquid formulation for oral administration may include suspensions, solutions, emulsions, syrups, and the like. In such embodiments, various excipients such as wetting agents, sweetening agents, fragrances, preservatives, and the like may be included in addition to water and liquid paraffin which are commonly employed simple diluents. A formulation for parenteral administration may include sterilized aqueous solutions, non-aqueous solutions, suspensions, emulsions, lyophilized formulations, and suppositories. In such embodiments, examples of non-aqueous solutions or suspensions include propylene glycol, polyethylene glycol, vegetable oil such as olive oil, an injectable ester such as ethyl oleate, and the like. Examples of suppository bases include glycerides, laurin fat, cacao butter, glycerol-gelatin, and the like.

In some embodiments, the composition has a pH of 1, 2, 3, 4, 5, 6, 7, 8, 9, 10, 11, 12, 13, or 14. In some embodiments, the composition has a pH in the range of about 4 to about 8, or in the range of about 5 to about 7. In some embodiments, the composition has a pH in the range of about 5.5 to about 6.

In some embodiments, the viscosity of the composition is selected to achieve a desired result (e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000 cps, etc., as measured on a Brookfield Viscometer using a TC spindle at 2.5 rpm at 25° C.). In some embodiments, the composition has a predetermined viscosity to assist in the administration of the composition and/or to avoid significant dripping or pooling after administration.

The dose of the herbal composition is not particularly limited and may be appropriately selected depending on the condition and body weight of patient, the severity of disease or condition, the drug form, and the route and period of administration. For example, the herbal composition may be administered in an amount ranging from 0.0001 to 1000 g/kg per day on a dry weight basis, or may be 0.001 to 100 g/kg per day, 0.001 to 10 g/kg per day, 0.001 to 1 g/kg per day, 0.0001 g/kg or more per day, 0.001 g/kg or more per day, 0.05 g/kg or more per day, 0.01 g/kg or more per day, or 0.005 g/kg or more per day.

In some embodiments, the herbal composition comprises one or more carriers, excipients, diluents, stabilizers, lubricants, moisturizers, penetration/permeation enhancers, and/or solubilizers, such as those used for various routes of intranasal administration. In such embodiments, diluents and excipients, such as fillers, extenders, binders, thickening agents, emulsifying agents, structuring agents, wetting agents, lubricants, disintegrants, or surfactants, may be used. In some embodiments, the composition comprises one or more pharmaceutical agents, silicone compounds, antioxidants, essential oils, botanical extracts, cosmetic components, preservatives, probiotics, probiotic derivatives, or prebiotics. The compositions may optionally contain a permeation enhancer to increase the rate at which the herbal composition permeates through the skin or mucosal tissue.

Carriers and Formulations

In some embodiments, the composition comprises an acceptable carrier (also referred to as a vehicle). Example carriers include water, glycerin, ethanol, chlorphenesin, sodium metabisulfite, oil (e.g., olive oil, coconut oil), or a combination thereof.

In some embodiments, the composition can be incorporated into one or more types of carriers. Examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art. Variations and other appropriate vehicles would be apparent to one of ordinary skill in the art. In some embodiments, the concentrations and combinations of the compounds, components, and agents are selected such that the combinations are chemically compatible and do not form complexes that precipitate from the finished product.

Moisturizing Agents

In some embodiments, the composition comprises one or more moisturizing agents. Examples of moisturizing agents include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. Further examples include acetylated lanolin, acetylated lanolin alcohol, alanine, algae extract, aloe barbadensis, aloe-barbadensis extract, aloe barbadensis gel, allantoin, althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, Calophyllum inophyllum (foraha) nut oil, candelilla (Euphorbia cerifera) wax, canola oil, caprylic/capric triglyceride, cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, clove leaf oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, emu oil, erythritol, ethoxydiglycol, ethyl linoleate, eucalyptus globulus oil, evening primrose (Oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myri state, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, pomegranate seed oil (Unica granatum), potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (Oryza sativa) bran oil, RNA, rosemary (Rosmarinus officinalis) oil, rose oil, rosehip seed oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tea tree oil (Malaleuca alternifolia), tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

Antioxidants

In some embodiments, the composition comprises one or more antioxidant agents. Examples of antioxidants include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphit.

Thickening Agents

In some embodiments, the composition comprises one or more thickening agents. As used herein, a thickening agent, or thickener, or gelling agent, includes one or more substances that can increase the viscosity of the composition. In some embodiments, thickeners increase the viscosity of the composition without substantially modifying the efficacy of the one or more active components within the composition. In some embodiments, thickeners increase the stability of the composition. In some embodiments, thickeners include hydrogenated polyisobutene or trihydroxystearin, or a mixture of both.

In various embodiments, thickening agents include carboxylic acid polymers, crosslinked polyacrylate polymers, polyacrylamide polymers, polysaccharides, and gums. Examples of crosslinked polyacrylate polymers include cationic and nonionic polymers.

Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Further examples of carboxylic acid polymers include carbomers, which are homopolymers of acrylic acid crosslinked with allyl ethers of sucrose or pentaerytritol.

Examples of polyacrylamide polymers (including nonionic polyacrylamide polymers having substituted, branched, or unbranched polymers) include polyacrylamide, isoparaffin and laureth-7, multi-block copolymers of acrylamides and substituted acrylamides with acrylic acids and substituted acrylic acids.

Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Further examples include an alkyl-substituted cellulose in which one or more of the hydroxy groups of the cellulose polymer is hydroxyalkylated (e.g., hydroxy ethylated or hydroxypropylated) to form a hydroxyalkylated cellulose that can then be modified with a C₁₀-C₃₀ straight chain or branched chain alkyl group through an ether linkage. In some embodiments, the polymers are ethers of C₁₀-C₃₀ straight or branched chain alcohols with hydroxyalkylcelluloses. Further examples of polysaccharides include scleroglucans comprising a linear chain of (1-3) linked glucose units with a (1-6) linked glucose every three unit.

Examples of gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Emulsifying Agents

In some embodiments, the composition comprises one or more emulsifiers. As used herein, an emulsifier is an agent that can reduce the interfacial tension between phases and improve the formulation and stability of an emulsion. The emulsifiers can be nonionic, cationic, anionic, and zwitterionic emulsifiers. Examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof

Surfactants

In some embodiments, the composition comprises a surfactant. As used herein, a surfactant is a compound that reduces surface tension between two liquids, or between a liquid and a solid. Surfactants can also function as cleaning agents, wetting agents, emulsifiers, foaming agents, and dispersants. Examples of surfactants include coco glucoside, ethoxylated fatty alcohol ethers, PEG castor oil, PEG esters, propylene glycol esters, glyceryl esters and derivatives, polymer ethers, sorbitan derivatives, and aliphatics. Examples include alcohols, emulsified waxes, and mixtures thereof. Exemplary ethoxylated aliphatic alcohol ethers are not particularly limited, but are limited, but are not limited to: steareth-2, steareth-10, steareth-20, steareth-21, steareth-40, steareth-100, behenes-10, ceteares-2, and ceteares.-3, Ceteares-5, Ceteares-6, Ceteares-10, Ceteares-12, Ceteares-15, Ceteares-20, Ceteares-21, Ceteares-22, Ceteares-25, Ceteares-30, Ceteares-31, Ceteares-32, Ceteares-33, Setes-2, Setes-10, Setes-20, Setes-23, Corres-24, Isocetes-20, Laureth-2, Laureth-3, Laureth-4, Laureth-5, Laureth-9, Laureth-10, Laureth-12, Laureth-15, Laureth-20, Laureth-21, Laureth-22, Laureth-23, Nonoxinol-9, Nonoxinol-15, Octoxinol-1, Octoxinol-9, Oles-2, Oles-5, Oles-10, Oles-20, C20-40 Palace-24 and Trideces-10, and mixtures thereof. Exemplary PEG castor oils include PEG-7 hydride castor oil, PEG-25 hydride castor oil, PEG-30 castor oil, PEG-33 castor oil, PEG-35 castor oil, PEG-36 castor oil, PEG-40 castor oil, PEG-40 hydride castor oil, PEG-50 castor oil, PEG-54 hydride castor oil, PEG-60 castor oil and PEG-60 hydride castor oil, and mixtures thereof. Can be mentioned. Exemplary PEG esters are not particularly limited, but are PEG-4 dilaurate, PEG-150 distearate, PEG-12 glyceryl laurate, PEG-120 glyceryl stearate, PEG-6 isostearate, PEG-4 laurate., PEG-8 laurate, PEG-20 methyl glucose sesquistearate, PEG-5 oleate, PEG-6 oleate, PEG-10 oleate, PEG-25 propylene glycol stearate, PEG-2 stearate, stearic acid PEG-6, PEG-6-32 stearate, PEG-8 stearate, PEG-9 stearate, PEG-20 stearate, PEG-40 stearate, PEG-45 stearate, PEG-50 stearate and stearic acid PEG-100, as well as mixtures thereof. Exemplary propylene glycol esters include propylene glycol laurate, propylene glycol palmitostearate, propylene glycol ricinoleic acid and propylene glycol stearate, and mixtures thereof. Exemplary glyceryl esters and derivatives include glyceryl behenate, glyceryl dibechenate, glyceryl dioleate, glyceryl distearate, glyceryl isostearate, glyceryl laurate, glyceryl linoleate, glyceryl monostearate, oleic acid. Glyceryl, glyceryl palmitate, glyceryl lysinolate, glyceryl stearate, PEG-23 glyceryl cocoate, PEG-6 caprylic acid/glyceryl caprate, PEG-7 glyceryl cocoate, polyglyceryl distearate-10, polyglyceryl diisostearate-2, polyglyceryl hypostearate-3 and polyglyceryl diisostearate-6, PEG-12 glyceryl laurate, PEG-120 glyceryl stearate, and mixtures thereof. Exemplary polymer ethers include poloxamer 124, poloxamer 181, poloxamer 182, poloxamer 184, poloxamer 188, poloxamer 237, poloxamer 331, poloxamer 338 and poloxamer 407, and mixtures thereof Exemplary sorbitan derivatives include polysorbate 20, polysorbate 40, polysorbate 60, polysorbate 65, polysorbate 80, sorbitan monolaurate, sorbitan monooleate, sorbitan monopalmitate, sorbitan monostearate, and sorbitan sesqui. Includes oleate, sorbitan trioleate and sorbitan tristearate, and mixtures thereof. Exemplary aliphatic alcohols include isostearyl alcohols, caprylyl alcohols, decyl alcohols, lauryl alcohols, myristyl alcohols, behenyl alcohols, lanolin alcohols, arachidyl alcohols, oleyl alcohols, palm alcohols, isosetyl alcohols, cetyl alcohols, stearyl alcohols, and cetearyl alcohols, and mixtures thereof. Exemplary emulsifying waxes include a mixture of cetearyl alcohol and polysorbate 60.

Structuring Agents

In some embodiments, the composition comprises a structuring agent. As used herein, a structuring agent modifies the rheological characteristics of the composition to contribute to the composition's stability. In some embodiments, the structuring agent also functions as an emulsifier or surfactant. Examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof

Preservatives

In some embodiments, the composition comprises one or more preservatives. Examples of preservatives include quaternary ammonium preservatives, such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, or combinations thereof

Essential Oils

In some embodiments, the composition comprises one or more essential oils. Essential oils include oils derived from herbs, flowers, trees, and other plants. Such oils can be present as tiny droplets between the plant's cells and can be extracted by methods known to those of skill in the art (e.g., steam distilled, enfleurage (i.e., extraction by using fat), maceration, solvent extraction, or mechanical pressing). When essential oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 to 240° C. and densities ranging from about 0.759 to about 1.096.

Essential oils are generally named by the plant from which the oil is found. For example, rose oil or peppermint oil are derived from rose or peppermint plants, respectively. Examples of essential oils include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Botanical Extracts

In some embodiments, the composition comprises one or more botanical extracts. Botanical extracts are derived from various parts of herbs, trees, and other plants including, the flowers, stems, seeds, roots, and fruit thereof. In some embodiments, the extracts include one or more of the following: absinthium, aconite, agar, algin, allantoin, aloe vera, althea root, ambrette seed, angelica, anise, Arnica Montana, asafetida, astragalus, avocado, lemon balm, barberry, basil sweet, bamboo (Bambusa vulgaris), bayberry bark, bee pollen, beeswax, benzoin, bilberry, bisabolol, blood root, Boswelia serrata, borage, buckthorn, buckwheat honey, burdock, cade oil, calamus, calendula, cananga oil, caraway, cardamon, cascara sagrada, cascarilla bark, celery seed, chamomile, chaparral, chenpodium, chicory root, coatis (Rhizoma coptidis), comfrey root, Cortex phellodendri chinensis (amur cork tree bark) dandelion root, Dictamnus root bark (Cortex dictamni) echinacea, eleuthero, eucalyptus, evening primrose, fangfeng, fenugreek, feverfew, Fo-ti, ganoderma, genet, geranium (wild), ginko, ginseng, gotu kola, giant knotweed (Rhizoma polygonie cuspidate) rhizome honey, hops, horse chestnut, horsetail, hypericum, Irish moss, job's tears, jojoba, jujube, licorice root, lycium fruit, magnolia flower, melatonin, neem, parsley, Pisum sativum (pea) seed extract, poria, quillaia, radix sophorea flavescetis (light yellow spoor root), rehmannia, rhoiola rosea root, rose hips, rosemary, rubarb root (Rhizoma rhei), sage, sarsaparilla, sea kelp, tannic acid, thyme, willow bark, witch hazel, yarrow, and yucca.

Additional Herbal Extracts

In some embodiments, the composition comprises one or more additional plant extracts. In some embodiments, the extracts are from plants used in traditional Chinese medicine for their healing, anti-swelling, wound healing, anti-inflammatory, or antihistaminic properties. In some embodiments, the herbs can include: atractylodes (baizhu and cangzhu), baizhu (atractylodes), bletilla tuber (baiji), cangzhu (atractylodes), dahurian angelica (baizhi), dittany bark (baixianpi), forsythia fruit (lianqiao), garden burnet (diyu), honeysuckle flower (jinyinhua), knotweed, giant (huzhang), ligusticum (gaoben), luffa (sigualuo), mume (smoked plum or wumei), pearl (zhenzhu or margarita), mother of pearl (zhenzhumu), peony bark and peony root (mudapi, shaoyao: chishaoyao and baishoyao), phellodendron bark (hungbai, purslane, common, machixian), red sage (danshen), safflower (false saffron: honghua), sichuan lovage (chuanxiog), skullcap, and baikal (huangqin).

Pharmaceutical Agents

In some embodiments, the composition comprises one or more pharmaceutical agents. Examples of pharmaceutical agents include analgesics, anesthetics, antihistamines, anti-inflammatory agents including non-steroidal anti-inflammatory drugs, antibiotics, antifungals, antivirals, antimicrobials, antipruritics, antipsoriatic agents, antiseborrheic agents, biologically active proteins and peptides, barrier agents, corticosteroids (e.g., hydrocortisone), wound treatment agents, and wound healing agents.

In some embodiments, the herbal composition comprises, in final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99%, or any range derivable therein, of at least one of the components mentioned throughout this disclosure. The percentage can be calculated by weight or volume of the total composition. In some embodiments, the concentrations vary depending on the addition, substitution, and/or subtraction of one or more components in a respective composition.

The various ingredients above are listed in specific categories (e.g., carrier, moisturizer, structuring agent, etc.) for convenience only. In some embodiments, a particular ingredient may be included in the topical composition for multiple reasons or functions. For example, the topical composition may include a particular ingredient for its properties and ability to function as a carrier and as a moisturizer. Any ingredient listed under one specific category may be used for an alternative purpose, and no unnecessary limitations should be read into the claims based on the specific categories.

### EXAMPLES

The following examples are provided to aid in the understanding of the present disclosure, the true scope of which is set forth in the appended claims. One of skill in the art would appreciate that modifications can be made in the procedures set forth without departing from the spirit of the disclosure.

Method of Preparing Herbal Compositions.

In this example, an herbal composition comprising Lonicera Japonica, Andrographis Paniculata, aloe vera, and melatonin was prepared according to the following steps.

Steam Distillation and Aloe Vera Hydrosol. 700 grams of organic aloe barbadensis was cut into 1-millimeter-wide slices were subjected to steam distillation. The plant material was placed into a distillation vessel (LT3000) and steam was generated from the deionized water contained in the boiler. The steam passed from the boiler, through the plant matter column, across a manifold pipe, and through the cooled condenser into a collection vessel. This process was run until about 500 milliliters of hydrosol (herbal distillate) was collected.

Herbal Extracts. An extract was prepared from Andrographis paniculata and Lonicera japonica flos. A cold ethanol extraction method was used to minimize lipid and chlorophyll contamination in the final product. The solvent was cooled to −40° C. before being combined with pre-frozen plant material at a ratio of 1:1:10 Andrographis paniculata to Lonicera japonica flos to ethanol by weight. The maceration was then placed in a sealed reaction vessel and left for 12 hours at −30° C. The maceration was next passed through a wide, 200-micron filter and transferred into the evaporation flask of a rotary evaporator. The solvent was then removed, and the extracted residue was manually collected from the flask.

Combination 1 (for evening administration). Melatonin and hydroxypropyl-beta-cyclodextrin were combined in a reaction vessel with the aloe vera hydrosol and stirred for 20 minutes to form a solution. The solution concentration of melatonin and hydroxypropyl-beta-cyclodextrin were 0.052% (w/w) and 0.520% (w/w), respectively. The extracted herbal residue was then added to the solution in an amount such that its concentration was 0.666% (w/w) and then stirred for 20 additional minutes.

Combination 2 (for daytime administration). In a reaction vessel, hydroxypropyl-beta-cyclodextrin was combined with the aloe vera hydrosol and stirred for 20 minutes to form a solution. The solution concentration of hydroxypropyl-beta-cyclodextrin was 0.520% (w/w). The extracted herbal residue was then added to the solution in an amount such that its concentration was 0.666% (w/w) and then stirred for 20 additional minutes.

Method of Administering Herbal Composition to a Subject.

A nasal spray formulation comprising the herbal composition according to Combination 1 and Combination 2 were prepared for intranasal administration and administered to several patients. A sample set of those patients are included in the table below, along with a profile of the subject and the outcome of the intranasal treatment.

| No. | Subject Description and Results | Outcome |
|---|---|---|
| 1 | The subject is a registered nurse (RN) who used the nasal sprays twice | + |
| | daily while working in a busy inpatient Hospice Unit. The subject did not | |
| | acquire Covid-19 or any other respiratory viruses during the six months of | |
| | observation. During the observation period, the subject traveled from | |
| | Florida to Texas and used the nasal sprays while staying with his family | |
| | members. After that visit, all family members tested positive for Covid-19 | |
| | except him and his partner, who used the nasal sprays three times per day | |
| | during the long weekend visit. | |
| 2 | The subject had severe radiation burns resulting from 48 radiation | + |
| | treatments to his head and neck cancer. He had green pus exudation from | |
| | his right eye and right nostril due to a chronic infection that was treated | |
| | with multiple courses of antibiotics, steroidal nasal sprays, and eye drops. | |
| | Despite those treatments, the infection persisted. The subject also had | |
| | blindness in his right eye due to the extensive radiation treatment. After | |
| | one week using the nasal sprays twice per day, the pus exudation stopped | |
| | in his eye and nose, and the infection cleared up. After one month of using | |
| | the nasal sprays, day and night, his eyesight started to return. The subject | |
| | continues to use the nasal sprays and has reported marked improvement in | |
| | his eyesight with no untoward side effects. | |
| 3 | The subject is a Doctor of Oriental Medicine and uses the nasal sprays | + |
| | while treating patients and while home with extended family members. | |
| | The subject has treated many Covid-19 patients during the pandemic and | |
| | has not acquired the virus. | |
| 4 | The subject is an herbalist and nutritionist who treats patients daily. She | + |
| | has chronic and severe sinus infections (MRSA and Staph) that required | |
| | two previous sinus irrigation surgeries. She also has been prescribed | |
| | numerous courses of antibiotics and pharmaceutical nasal rinses. After six | |
| | months of using the nasal sprays, she reports a significant improvement in | |
| | her chronic sinus infections. Further, she has not acquired Covid-19 or any | |
| | other respiratory virus during the observation period. | |
| 5 | The subject is an executive officer at a hospital and has had chronic sinus | + |
| | infections with postnasal drip and chronic coughing. The subject has been | |
| | prescribed several courses of antibiotics and steroidal sprays, which failed | |
| | to provide relief of the infections and systems. After using the nasal spray | |
| | for only two nights of treatment, the subject reports the postnasal drip had | |
| | halted and his chronic cough was eliminated. | |
| 6 | The subject is the registered nurse (RN) manager of a hospital and was | + |
| | diagnosed with pneumonia and a positive Covid-19 Test. After using the | |
| | nasal sprays for one week, her clinical manifestations dramatically | |
| | improved and she credited the nasal sprays. After days of using the nasal | |
| | sprays, the subject reported a negative PCR test for Covid-19. | |
| 7 | The subject is a Doctor of Oriental Medicine with asthma and pulmonary | + |
| | fibrosis, and who has treated patients with Covid-19 over the last two | |
| | years. The subject reported use of the nasal sprays twice daily and | |
| | whenever she felt the onset of a respiratory virus. The subject has not had | |
| | any serious respiratory infections during the observation period. | |
| 8 | The subject has chronic insomnia and uses the nasal spray with melatonin | + |
| | daily in the evening. The subject reports an excellent improvement in her | |
| | sleep, perhaps the best sleep she has had during her adult life. The subject | |
| | reports that her husband started using the melatonin nasal spray at night | |
| | after observing his wife's improvement and he reports similar results. | |
| 9 | The subject is an executive officer at a large hospital. He used the nasal | + |
| | sprays daily in the morning and evening. During a six-month period, the | |
| | subject had numerous hospital meetings with other hospital employees | |
| | and has not acquired Covid-19 or any other respiratory virus during the | |
| | observation period. | |
| 10 | The subject is an assistant film director who has had Covid-19 twice from | + |
| | his exposures at work. The subject has used the nasal sprays for the six | |
| | months and has not tested positive for Covid-19, despite multiple | |
| | exposures to colleagues who tested positive for Covid-19 during the | |
| | observation period. | |
| 11 | The subject suffers from type-II diabetes, hypertension, metabolic | + |
| | syndrome, and long Covid-19. She had been treated with several courses | |
| | of antibiotics, anti-inflammatorys, steroids, and nasal sprays. The subject | |
| | reports long Covid symptoms for over six months, including a loss of | |
| | taste, a loss of smell, malaise, and brain fog. After three days using the | |
| | nasal sprays, the subject reports that her sense of taste and smell have | |
| | returned, and her malaise and brain fog have cleared. | |
| 12 | The subject is a 65-year-old male who contracted Covid-19. The subject | + |
| | started using the nasal sprays one week after his first Covid symptoms | |
| | appeared. The subject reports that his sense of smell and taste returned | |
| | after one day of using the nasal sprays. | |
| 13 | The subject is a 29-year-old male who runs product promotions, which | + |
| | involves distributing samples to the public at conventions, bars, and | |
| | restaurants. The subject has used the nasal sprays twice per day for six | |
| | months and has not acquired Covid-19 during the observation period. | |

Legend: + indicates a positive subject outcome, with no reported negative side effects.

A recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a composition having 1-3 components refers to compositions having 1, 2, or 3 components. Similarly, a composition having 1-5 components refers to compositions having 1, 2, 3, 4, or 5 components, and so forth.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” and “substantially” will be understood by persons of ordinary skill in the art and will vary to some extent depending upon the context in which it is used. If there are uses of the term which are not clear to persons of ordinary skill in the art, given the context in which it is used, “about” and “substantially” will mean up to plus or minus 10% of the particular term.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, avis, etc. In some embodiments herein, the mammal is human.

The term “administering” a composition to a subject means delivering the composition to the subject. “Administering” can include prophylactic administration of the composition (i.e., before one or more symptoms of a respiratory disease or condition are detectable) and/or therapeutic administration of the composition (i.e., after the respiratory disease or condition and/or one or more symptoms of the respiratory disease or condition are detectable). The methods of some embodiments may include administering one or more compounds, compositions, or agents. If more than one compound, composition, or agent is to be administered it may be administered together at substantially the same time, and/or be administered before, concomitantly with, and/or after administration of another composition or therapeutic procedure (e.g., acupuncture).

As used herein, the terms “effective amount” or “therapeutically effective amount,” refer to a quantity sufficient to achieve a desired therapeutic and/or prophylactic effect, e.g., an amount which results in the full or partial amelioration of disorders or symptoms, e.g., those associated with respiratory disease or condition in a subject in need thereof. In the context of therapeutic or prophylactic applications, the amount of a composition administered to the subject will depend on the type and severity of the disorder or symptom and on the characteristics of the individual, such as general health, age, sex, body weight, and tolerance to drugs or herbs. It will also depend on the degree, severity, and type of disorder or symptom. The compositions can also be administered in combination with one or more additional compounds, compositions, or herbs. In some embodiments, multiple doses are administered. In some embodiments, multiple therapeutic compositions or compounds are administered. In the methods described herein, the compositions may be administered to a subject having one or more signs or symptoms of a disorder described herein.

Exemplary embodiments of the herbal compositions and methods are described above in detail. The herbal compositions and methods are not limited to the specific embodiments described herein, but rather, components of the herbal compositions and/or steps of the method may be utilized independently and separately from other components and/or steps described herein.

This written description uses examples to disclose the present embodiments, including the best mode, and also to enable any person skilled in the art to practice the present embodiments, including making and using any herbal compositions or performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I/We claim:

1. An herbal composition, comprising:
Lonicera Japonica and Andrographis Paniculata;
wherein each of the Lonicera Japonica and Andrographis Paniculata is included, individually, in an amount ranging from about 0.01% to about 5% w/w.

2. The herbal composition of claim 1, wherein each of the Lonicera Japonica and Andrographis Paniculata is included, individually, in an amount ranging from about 0.1% to about 1% w/w.

3. The herbal composition of claim 1, wherein the Lonicera Japonica and Andrographis Paniculata have a combined concentration of 0.666% (w/w).

4. The herbal composition of claim 1, wherein the Lonicera Japonica and Andrographis Paniculata are included in a concentration ratio in the range of 0.2 to 5.0.

5. The herbal composition of claim 1, further comprising melatonin.

6. The herbal composition of claim 1, further comprising aloe vera.

7. The herbal composition of claim 1, further comprising hydroxypropyl-beta-cyclodextrin.

8. The herbal composition of claim 7, further comprising melatonin;
wherein the melatonin has a concentration of 0.052% (w/w) and the hydroxypropyl-beta-cyclodextrin has a concentration of 0.520% (w/w).

9. The herbal composition of claim 1, wherein the herbal composition is formulated for intranasal administration.

10. A method of treating a respiratory or inflammatory condition in a subject in need thereof, comprising:
administering an herbal composition to the subject, the herbal composition comprising Lonicera Japonica and Andrographis Paniculata;
wherein each of the Lonicera Japonica and Andrographis Paniculata is included, individually, in an amount ranging from about 0.01% to about 5% w/w.

11. The method of claim 10, wherein each of the Lonicera Japonica and Andrographis Paniculata is included, individually, in an amount ranging from about 0.1% to about 1% w/w.

12. The method of claim 10, wherein the Lonicera Japonica and Andrographis Paniculata have a combined concentration of 0.666% (w/w).

13. The method of claim 10, wherein the Lonicera Japonica and Andrographis Paniculata are included in a concentration ratio in the range of 0.2 to 5.0.

14. The method of claim 10, wherein the herbal composition further comprises aloe vera.

15. The method of claim 10, wherein the herbal composition further comprises melatonin.

16. The method of claim 10, wherein the herbal composition further comprises hydroxypropyl-beta-cyclodextrin.

17. The method of claim 16, wherein the herbal composition further comprises melatonin;
wherein the melatonin has a concentration of 0.052% (w/w) and the hydroxypropyl-beta-cyclodextrin has a concentration of 0.520% (w/w).

18. The method of claim 17, wherein the herbal composition further comprises hydroxypropyl-beta-cyclodextrin.

19. The method of claim 17, wherein the melatonin has a concentration of 0.052% (w/w) and the hydroxypropyl-beta-cyclodextrin has a concentration of 0.520% (w/w).

20. A method of treating a respiratory or inflammatory condition in a subject in need thereof, comprising:
administering an herbal composition to the subject, the herbal composition comprising Lonicera Japonica and Andrographis Paniculata;
wherein each of the Lonicera Japonica and Andrographis Paniculata is included, individually, in an amount ranging from about 0.01% to about 5% w/w;
wherein the Lonicera Japonica and Andrographis Paniculata are included in a concentration ratio in the range of 0.2 to 5.0; and
wherein the administering is intranasal administration.
